(19) United States
(12) Patent Application Publication
(10) Pub. No.: US 2016/0223534 A1
(43) Pub. Date: Aug. 4, 2016

(54) DEVICE AND METHOD FOR DETECTING, WHILE USING A MICROFLUIDIC CHIP, THE RESISTANCE OF BACTERIA TO AN ACTIVE SUBSTANCE TO BE ANALYZED

(71) Applicant: Fraunhofer-Gesellschaft zur Foerderung der angewandten Forschung e.V., Munich (DE)

(72) Inventors: Jennifer SCHMIDT, Falkenstein (DE); Anna OHLANDER, Muenchen (DE)

(21) Appl. No.: 15/016,003

(22) Filed: Feb. 4, 2016

(30) Foreign Application Priority Data
Feb. 4, 2015 (EP) 15153843.6
Feb. 10, 2015 (DE) 102015202353.2

(51) Int. Cl. G01N 33/543; G01N 33/58; C12Q 1/18

(52) U.S. Cl. CPC G01N 33/54366; C12Q 1/18; G01N 33/582

(57) ABSTRACT

A device for detecting resistant bacteria includes a microfluidic chip, a detector and an analyzer. The microfluidic chip includes fluidic structures having a fluid inlet and an interaction chamber fluidically connected to the fluid inlet, and a labeled substrate that is immobilized inside the interaction chamber and enters into a verifiable interaction with bacterial factors of bacteria, so that by introducing a culture medium containing an active substance to be analyzed and the bacteria into the interaction chamber and by detecting the result of the verifiable interaction, it can be ascertained whether the bacteria are resistant to the active substance to be analyzed. The detector detects information that is a measure of whether or not the verifiable interaction has taken place. On the basis of the detected information, the analyzer outputs a display regarding the resistance of the bacteria to the active substance to be analyzed.

### CROSS-REFERENCE TO RELATED APPLICATIONS

This application claims priority to European Patent Application No. 15153843.6, which was filed on Feb. 4, 2015, as well as claims priority to German Patent Application No. 102015202353.2 filed Feb. 10, 2015, both of which are incorporated herein in entirety by reference.

The present invention relates to devices and methods for detecting, while using a microfluidic chip, whether bacteria are resistant to an active substance to be analyzed.

### BACKGROUND OF THE INVENTION

The large-scale utilization of antibiotics, for example in the fields of medicine and animal fattening, of agriculture and cattle farming, as well as their improper use result in that over the years, bacteria have developed pronounced resistances to the active substances employed. Infectious diseases are the cause of more than ten millions deaths per year, but the rapid build-up of resistance of many pathogens increasingly restricts therapeutic options.

A particularly large amount of infections are triggered by resistant strains of the species Staphylococcus aureus. In the time period from 1999 to 2004, for example, the proportion of methicillin-resistant staphylococci (MRSA) rose to a constant 20 percent. This problematic germ is frequently the cause of nosocomial infections in hospitals, but is also often found in facilities were elderly, immunodeficient or immunosuppressed persons are accommodated, such as old people's homes and nursing homes, for example. In addition to Staphylococcus aureus, there are by now a multitude of further resistant bacteria (such as enterococci, pneumococci, Pseudomonas aeruginosa, Campylobacter, EHEC, and others) whose infectious diseases are very hard to treat.

Testing of sensitivities of bacteria in terms of new potential active substances is currently performed in various methods requiring cultural propagation of the bacteria, which involves a large amount of time and cost, however, since said cultivation is effected under specific conditions, over several hours, regarding air humidity, CO₂, incubation temperature and time.

There are various methods of determining the in-vitro sensitivity of bacteria to specific substances, such as the agar diffusion test, the Etest, and serial dilution tests such as broth microdilution or agar dilution, for example.

Broth Dilution Test/Serial Dilution Test

Serial dilution tests may be performed while using liquid nutrient media (nutrient broth) in test tubes (macrodilution) and microtiter plates (microdilution). In addition, solid nutrient media, too, are used for agar dilution, which is not suitable for performing active-substance screenings, however, since large amounts of substances are used and the method is very costly.

The method most frequently used for active-substance screening is the macrodilution method. Here, a defined bacterial count is incubated in the presence of different concentrations of the active substance to be analyzed. The lowest concentration of active substance at which visible growth of the bacteria can no longer be ascertained is referred to as the minimal inhibitory concentration (MIC). Growth is ascertained by means of the turbidity of the medium. What is disadvantageous is that comparatively large amounts of active substance are employed, the method is relatively costly, and long incubation times are needed before visible turbidity of the medium occurs. In addition to the macrodilution method, there are also microdilution methods, which are employed in routine diagnostics for preparing antibiograms. For the purpose of simplified handling, microtiter plates are already commercially available for this, Schwarz et al., “Empfindlichkeitsprüfung bakterieller Infektionserreger von Tieren gegenüber antimikrobiellen Wirkstoffen: Methoden zur invitro Empfindlichkeitsprüfung and deren Eignung in Hinblick auf die Erarbeitung therapeutisch nutzbarer Ergebnisse” (“sensitivity test of bacterial pathogens of animals with regard to antimicrobial active substances: methods for in-vitro sensitivity testing and their suitability with regard to working to obtaining results that can be used therapeutically”), Berl. Münch. Tierärztl. Wschr. (2003) 116, 353-361. MBC determinations (MBC=minimal bactericidal concentration) are possible by means of dilution tests; however, these are very labor-intensive to perform.

Agar Diffusion Test

In the agar diffusion test, the bacterial germ to be analyzed is homogenously applied to a solid nutrient medium (typically Müller-Hinton agar) in a defined bacterial count. The growth medium is covered with a platelet equipped with a defined amount of active substance. The active substance diffuses out of the platelet into the growth medium, wherein an active-substance gradient forms around the platelet. As a function of its sensitivity, the bacterial pathogen to be examined is prevented from growing within an area around the platelet, the size of said area differing. This area is referred to as an inhibiting areola. Depending on the size of the diameter of the inhibiting areola, qualitative classification of the bacteria in terms of “sensitive”, “intermediate”, or “resistant” is performed, see Schwarz et al. This method is not suitable for performing active-substance screening at a high throughput, however.

Epsilon Test (Etest)

The epsilon test (Etest) is an agar diffusion test allowing determination of MIC values. The active-substance carrier is a plastic strip containing an active-substance gradient, i.e. an ascending antibiotics concentration. Just like the platelet in the agar diffusion test, the strip is applied to a homogenously inoculated agar plate. Once an ellipsoid inhibiting areola has formed in the case of sensitive germs, the MIC value can be read off, at the point of intersection of this unpopulated zone, by means of the test strip by using a printed-on scale, as is described in Altreuther et al., “Anmerkungen zum Resistenzmonitoring in der Tiergesundheit” (“comments on resistance monitoring in animal health”); Berl. Munch. Tierärztl. Wschr. (1997) 110, pages 418-421. What is disadvantageous is that this test is comparatively expensive and that the antibiotics employed in veterinary medicine are not offered or offered only partly. This method, too, is unsuitable for screening new active substances.

Automated methods

For setting up antibiograms, i.e. test for determining the sensitivity or resistance of microbial pathogens to antibiotics, automated methods are employed which are known, e.g., by the names or brands of Mini-API, ATB, VITEK (Bio-Merieux), WalkAway (Dade-Behring) or Phoenix (Becton Dickinson). In many of those systems, evaluation takes place by means of the turbidity of the media that follows growth of resistant bacteria. This involves an incubation step which often takes several hours.

### SUMMARY

According to an embodiment, a device for detecting the resistance of bacteria to an active substance to be analyzed may have:

a microfluidic chip, which may have:
fluidic structures having a fluid inlet and an interaction chamber fluidically connected to the fluid inlet;
a labeled substrate that is immobilized inside the interaction chamber and is configured to enter into a verifiable interaction with bacterial factors of bacteria, so that by introducing a culture medium containing an active substance to be analyzed and the bacteria into the interaction chamber and by detecting the result of the verifiable interaction, it can be ascertained whether the bacteria are resistant to the active substance to be analyzed,
a void volume of the fluidic structures not exceeding 100 μl;

a detector configured to detect information that is a measure of whether or not the verifiable interaction has taken place; and

an analyzer configured to output, on the basis of the detected information, a display with regard to the resistance of the bacteria to the active substance to be analyzed.

According to another embodiment, a method of detecting the resistance of bacteria to an active substance to be analyzed may have the steps of: introducing a culture medium containing an active substance to be analyzed and bacteria into the interaction chamber of a microfluidic chip which has fluidic structures having a fluid inlet and an interaction chamber fluidically connected to the fluid inlet, and has a labeled substrate immobilized inside the interaction chamber and being configured to enter into a verifiable interaction with bacterial factors of bacteria, a void volume of the fluidic structures not exceeding 100 μl; detecting information that is a measure of whether or not the verifiable interaction has taken place; and outputting a display, on the basis of the detected information, with regard to the resistance of the bacteria to the active substance to be analyzed.

Embodiments provide a device for detecting the resistance of bacteria to an active substance to be analyzed, comprising:

a microfluidic chip comprising:
fluidic structures having a fluid inlet and an interaction chamber fluidically connected to the fluid inlet;
a labeled substrate that is immobilized inside the interaction chamber and is configured to enter into a verifiable (German: nachweisbare) interaction with bacterial factors of bacteria so that by introducing a culture medium containing an active substance to be analyzed and the bacteria into the interaction chamber and by detecting the result of the verifiable interaction, it can be ascertained whether the bacteria are resistant to the active substance to be analyzed,
a void volume of the fluidic structures not exceeding 100 μl;

a detector configured to detect information that is a measure of whether or not the verifiable interaction has taken place; and

an analyzer configured to output, on the basis of the detected information, a display with regard to the resistance of the bacteria to the active substance to be analyzed.

Embodiments provide a method of detecting the resistance of bacteria to an active substance to be analyzed, comprising:

introducing a culture medium containing an active substance to be analyzed and bacteria into the interaction chamber of a microfluidic chip which comprises fluidic structures having a fluid inlet and an interaction chamber fluidically connected to the fluid inlet, and comprises a labeled substrate that is immobilized inside the interaction chamber and is configured to enter into a verifiable interaction with bacterial factors of bacteria, a void volume of the fluidic structures not exceeding 100 μl;

detecting information that is a measure of whether or not the verifiable interaction has taken place; and

outputting a display, on the basis of the detected information, as to whether there are resistant bacteria.

Embodiments are based on the finding that it is possible, by using a microfluidic chip configured to deal with small volumes of liquid of an order of magnitude of 100 μl and below and by using a labeled substrate that is immobilized within the microfluidic chip, to detect the resistance of bacteria to an active substance to be analyzed while involving an amount of time that is reduced as compared to known methods and while using a small sample quantity.

### BRIEF DESCRIPTION OF THE DRAWINGS

Embodiments of the present invention will be detailed subsequently referring to the appended drawings, in which:

FIG. 1 shows a schematic representation of an embodiment of a device for detecting the resistance of bacteria to an active substance to be analyzed;

FIGS. 2 to 4 show schematic representations of microfluidic chips;

FIG. 5 shows a schematic representation for explaining an embodiment of a device and of a method for detecting the resistance of bacteria to an active substance to be analyzed; and

FIG. 6 shows a flow chart of an embodiment of a method described herein.

### DETAILED DESCRIPTION OF THE INVENTION

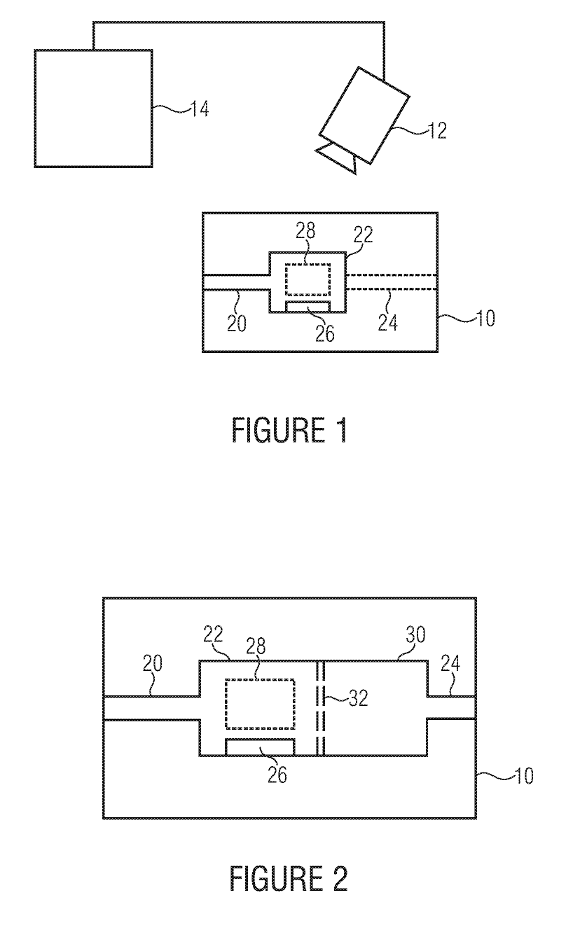

A schematic representation of a device for detecting the resistance of bacteria to an active substance to be analyzed is shown in FIG. 1. The device comprises a microfluidic chip 10, a detector 12, and an analyzer 14. The microfluidic chip 10 is represented as a schematic top view and comprises fluidic structures having an inlet 20 and a fluid chamber (interaction chamber) 22. Optionally, the fluidic structures also comprise an outlet 24 as shown by dashed lines in FIG. 1. In the figures, the fluid chamber is shown to have a diameter (in the flow direction from the inlet) that is larger than that of the inlet (or outlet). The fluid chamber 22 may also have the same diameter as that of the inlet (channel) 20 and the outlet (channel) 24. As is also shown by dashed lines in FIG. 1, a heating element 28 may be provided for heating the contents of the fluid chamber 22. The heating element 28 may be integrated into the microfluidic chip 10 or may be provided separately thereof. The heating element may be provided for locally heating the fluid chamber 22 and the contents thereof, or for globally heating the entire microfluidic chip 10. Heating elements as are described herein may be implemented, e.g., by using copper mesh heaters as are described by Ohlander et al., “Genotyping of single nucleotide polymorphisms by melting curve analysis using thin film semi-transparent heaters integrated in a lab-on-foil system”, Lab Chip (2013), 13, pages 2075-2082.

The labeled substrate is labeled with a substance and interacts with bacterial factors, said interaction being verifiable. In embodiments, the substrate may be labeled with fluorescent dyes (fluorophores). In alternative embodiments, the substrate may be marked with absorption dyes or radioactive substances. In embodiments, the interaction of the labeled substrate with the bacterial factors involves detaching the label or labeled substrate fragments from the labeled substrate. In embodiments, the interaction causes a change in color of the labeled substrate. In any case, the interaction changes a property of the labeled substrate that is detectable by the detector so as to detect, on the basis thereof, the resistance of bacteria to an active substance to be analyzed.

In addition to be fixedly bound to a wall of the fluid chamber, the labeled substrate, or the labeled substrates, may also be fixed to fibrous structures that are introduced into the reaction chamber and are immobilized, and/or to particulate systems that are introduced into the fluid chamber and are immobilized. For example, the fibrous structures and/or the particulate systems may be immobilized inside the fluid chamber by using a retention means. This entails the advantage of an increase in the surface area, and the substrates are possibly more sterically accessible to the bacterial factors.

Embodiments may be configured for active-substance screening in order to identify new potential substances having antimicrobial action against MRSA. Similarly, other species of bacteria may also be examined while using the approach described here; it is only useful to adapt the substrate immobilized inside the interaction chamber, which may also be referred to as a reaction chamber, to the bacteria being equipped with different bacterial factors. Said bacterial factors may be enzymes, for example. Once it is ensured that the cultures at hand are pure cultures, simple metabolic enzymes are also suitable in addition to virulence factors such as deoxyribonuclease, hyaluoronidase et al.

An active substance to be analyzed may herein be understood to mean a substance that has a potentially antimicrobial action. In particular, an active substance to be analyzed may also be understood to mean substances to be analyzed whose actions are not yet known in the course of the search for new anti-microbacterial active substances. Moreover, they may also be understood to mean antibiotics.

In accordance with embodiments of the invention, a microfluidic chip is thus employed for detecting the resistance of bacteria to an active substance to be analyzed. A microfluidic chip here is understood to mean a chip which comprises fluidic structures which may have dimensions within the millimeter or submillimeter ranges and are thus suited for dealing with very small volumes of fluid within the μl range.

In embodiments of the invention, the microfluidic chip may be a foil-based microfluidic chip built of different layers of plastic foils structured such that the fluidic structures result by placing the individual plastic foils (foil layers) on top of one another. The fluidic structures comprise an inlet and a fluid chamber. The fluidic structures may form a microfluidic channel comprising an inlet and an outlet, a central portion of the channel representing a fluid chamber. The plastic foils may be structured by means of lasers, for example.

In alternative embodiments, the microfluidic chip may also be implemented in a classic injection molding technique. However, utilization of plastic foils and the possibility associated therewith of using roll-to-roll manufacturing techniques instead of injection molding methods fundamentally offers a multitude of advantages. Elements such as heaters, for example, may be integrated, as is described by Ohlander et al. (2013), for example. It is also easily possible to immobilize labeled substrates or a labeled coating, as is described, for example, by Sun et al. “Direct immobilization of DNA probes on non-modified plastics by UV irradiation and integration in microfluidic devices for rapid bioassay”, Anal Bioanal Chem (2012), 402, pages 741-748. Low-cost production of said chips in very high numbers (e.g., on an industrial scale) can be easily performed. Since foil-based chips do not require any expensive molded articles to be pressed, they are easily adaptable to new conditions (or verification strategies), which is advantageous in the development of prototypes and offers highly flexible possibilities of application with regard to utilization at a later date.

During operation, a culture medium, the bacteria, and the active substance to be analyzed are introduced into the interaction chamber. In embodiments, a suspension containing a defined bacterial count, the active substance to be analyzed, and the culture medium may be introduced into the interaction chamber. Introduction may be effected in an automated manner, for example, it being possible for the inlet to be coupled to corresponding off-chip fluidic structures (not shown) for this purpose. Alternatively, said introduction may also be effected manually. Inside the interaction chamber, the suspension may be heated up to a temperature at which the interaction between the labeled substrate and the bacterial factors will take place if the bacterial factors are present, i.e., if the bacteria are resistant to the active substance to be analyzed. If the bacteria are not resistant (sensitive) or exhibit only little resistance (intermediate), the reaction will not take place or will take place to a small extent only. The detector is configured to detect information that is a measure of whether and, advantageously, to which extent the interaction has taken place. The detector provides said information to the analyzer, which may be configured to compare said information to one or several threshold values and to output a display, as a function of the comparison, as to whether or not the bacteria are resistant to the active substance to be analyzed. In embodiments, the analyzer may compare the information to a threshold value and may output, as a function of the comparison, a display stating that the bacteria are or are not resistant to the active substance to be analyzed. In embodiments, the analyzer may be configured to compare the information to several threshold values so as to enable a quantitative statement in terms of the resistance, for example as to whether the bacteria are resistant, intermediate, or sensitive to the active substance to be analyzed. For example, the bacteria will be evaluated as being resistant if the result shows that a first threshold value is not exceeded, will be evaluated as being intermediate if a second threshold value, which is higher than the first threshold value, is not exceeded, and will be evaluated as being sensitive if the second threshold value is exceeded. In embodiments, a larger number of threshold values may also be used.

The detector is configured to detect the respective interaction. In this context, the detector is adapted to the type of the labeled substrate and to the type of interaction. If fluorophores or absorption dyes are used as the labels, the detector may comprise a photomultiplier or a one-photon detector. Such detectors are highly sensitive to a fluorescent signal and may be capable of detecting even individual photons, which may enable reduction of the detection time. In particular, cultivation for several hours in order to multiply the sample quantities is not required. In embodiments, a silicon photomultiplier (SiPM) may be used for detection. Silicon devices are of interest because they can miniaturized, consume little energy while being low in cost. In alternative embodiments, high-resolution cameras may be used as the detectors. Depending on the type of the labeled substrate used, yet alternative embodiments may use detectors suitable for detecting radioactive radiation.

The analyzer may be implemented by a computing means, for example a computer, which is configured or programmed to analyze the information originating from the detector accordingly. In embodiments, the analyzer may comprise a processor, for example a microprocessor, which is coupled to a memory via a communication bus, the memory storing machine-readable commands which may be executed by the processor to provide the functionality described herein. The analyzer may further comprise a display device on which the result of the examination can be displayed. The display device may be a screen or a printer, for example.

In the embodiment shown in FIG. 1, the detector 12 may be arranged to detect a fluorescence occurring inside the interaction chamber 22. The labeled substrate may here be labeled with fluorophores and quenchers so that fluorescence will not occur until enzymatic cleavage occurs which is caused by bacteria which are located inside the interaction chamber 22 and are resistant to an active substance to be analyzed and/or by their bacterial factors. Thus, enrichment, reaction (interaction), and signal detection may simultaneously occur inside one single chamber. Such a setup enables performing dynamic measurements in a simple manner.

An embodiment wherein scattered-light effects caused by particles in the suspension can be prevented will now be explained with reference to FIG. 2, which shows a microfluidic chip 10 for such an embodiment. The fluidic structures of the microfluidic chip 10 shown in FIG. 2 comprise the inlet 20, the interaction chamber 22, a detection chamber 30 fluidically coupled to the interaction chamber 22 via a retention means 32, and the outlet 24. The retention means is configured to allow labels and/or substrate fragments, which are detached from the labeled substrate due to the interaction, to pass through and to retain the bacteria. The retention means may be implemented, for example, by a filter having a corresponding pore size (of e.g. 0.2 μm). In this embodiment, the labels of the labeled substrate may be formed by fluorophores, for example, which are detached from the substrate by the interaction.

In operation, a suspension may in turn be introduced into the interaction chamber 22 and be heated, as was described above. Subsequently, a flow through the fluidic structures may be caused in that a pressure is applied, for example, via the inlet 20, by an off-chip pump or an off-chip pressure transducer. Alternately, a pump or a pressure transducer may be integrated on the chip. In this manner, detached labels and/or labeled substrate fragments are driven through the retention means 32, while bacteria are retained inside the retention means 32. In this manner, the substances and/or labels which have passed through the retention means 32 can be detected inside the detection chamber 30 while using the detector (not shown in FIG. 2) directed to the detection chamber 30, without any scattered-light effects being caused by the bacteria or other particles that cannot pass through the retention means 32.

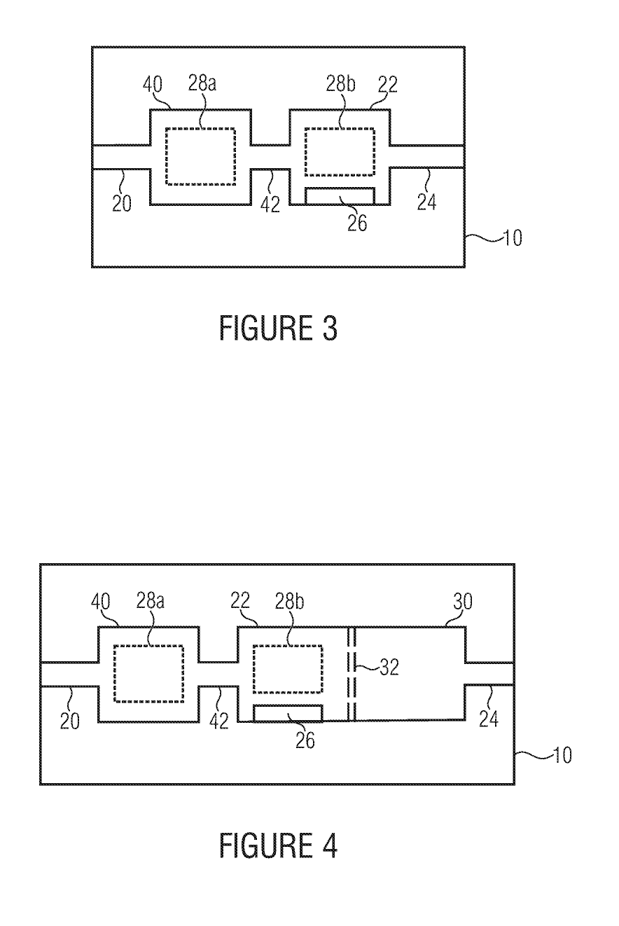

FIG. 3 shows a microfluidic chip 10 for an alternative embodiment, wherein the fluidic structures comprise an enrichment chamber 40 fluidically connected between the inlet 20 and the interaction chamber 22. The enrichment chamber 40 is connected to the interaction chamber 22 via a fluid channel 42. A first heating element 28a configured to heat the contents of the enrichment chamber and a second heating element 28b configured to heat the contents of the interaction chamber 22 are provided. The heating elements 28a and 28b may be provided in an on-chip or off-chip manner in each case. The heating element 28a is configured to heat the contents of the enrichment chamber to a temperature suitable for enriching the bacteria in the culture medium, and the heating element 28b is configured to heat the contents of the interaction chamber to a temperature suitable for the interaction.

In operation, a suspension may initially be introduced into the enrichment chamber 40, where it is heated up to an enrichment temperature suitable for cultivating the bacteria, e.g., 37° C. Following the cultivation, the suspension is pumped into the interaction chamber 22, for example by means of an on-chip or an off-chip pumping means. There the cultivated suspension may be heated up to a temperature suitable for the interaction, for example an enzymatic activity, such as, e.g., 45° C. Thus, it is possible to optimize the temperature for the respective operation. Detection of the interaction may then occur in the manner described above with reference to FIG. 1.

FIG. 4 shows a microfluidic chip, the fluidic structures of which comprise a combination of the features described with reference to FIGS. 2 and 3. Detection of information which is a measure of whether or not the verifiable interaction has taken place can be performed in the manner described with reference to FIG. 2.

In alternative embodiments, the microfluidic chip may comprise several substrates labeled with different labels and immobilized inside one or several fluid chambers of the microfluidic chip. It is thus possible to perform several different verification reactions (German: Nachweisreaktionen) in parallel on one chip. In order to be able to distinguish between the different verification reactions at the detector, different fluorescence labels (having different spectral properties) may be used for this purpose, for example. For example, a first bacterial factor A may release a fluorophore A, whereas a second bacterial factor B may release a fluorophore B, etc.

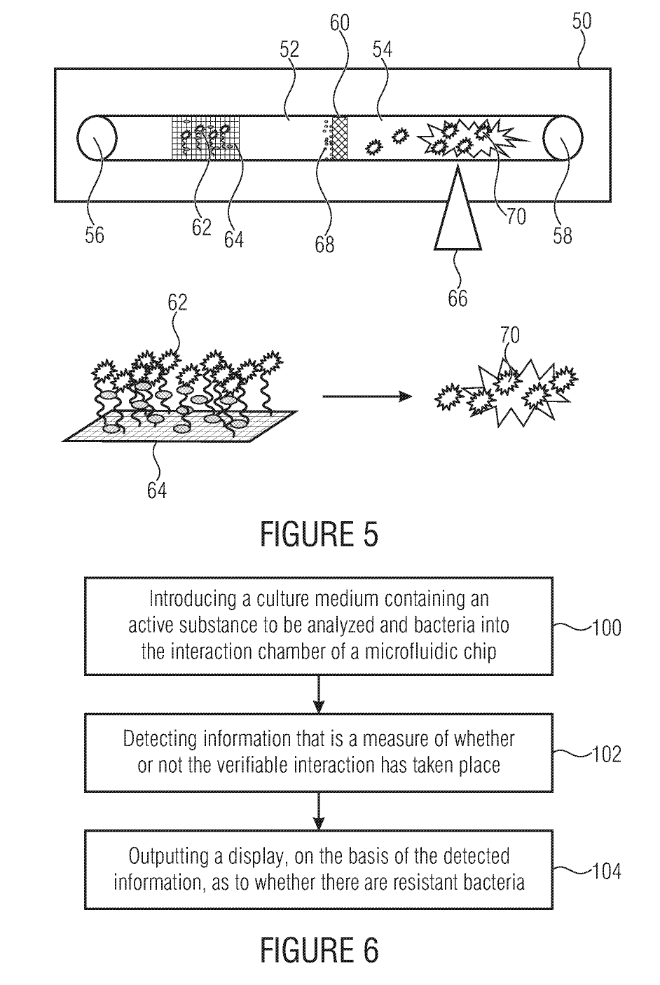

In the following, an embodiment will be described with reference to FIG. 5 by using the example of active-substance screening for identifying, while using a foil-based chip, new potential substances having anti-microbacterial actions against MRSA. The chip 50 comprises fluidic structures comprising two chambers, a first chamber 52 serving as an enrichment and reaction chamber (interaction chamber) and a second chamber serving as a detection chamber 54. In addition, the fluidic structures comprise an inlet 56 and outlet 58. The chambers 52 and 54 are connected to each other via a microfluidic channel, the chambers and the channel having an identical flow cross-section. The chambers 52 and 54 have a device 60, e.g., a filter membrane, located between them which serves to retain particles and bacteria from a sample stream between the chambers 52 and 54. DNA 62 (e.g., spotted pTpC oligonucleotides) immobilized on the foil and marked by a fluorescence label, see Sun et al., 2012, for example, is located in the area of the enrichment and reaction chamber 52. The rear side of the foil, on the front side of which the chamber 52 is formed, has the structure 64 of a heating element located thereon which may be a printed copper mesh heater, for example, which may be controlled via conductor lines and be thermostated accordingly, as described by Ohlander et al., 2013.

From that point of view, the chip 50 may comprise a setup as was described above with reference to FIG. 2.

If the sensitivity of MRSA strains to new, potentially antimicrobial active substances is to be analyzed, a defined count of MRSA bacteria is brought together with a defined concentration of the active substance to be tested, e.g., as a concentration series for determining MIC/MBC, within a small volume of culture medium. The overall volume of the chip (i.e., the volume available for being filled with fluid or liquid, which will also be referred to as a void volume herein, amounts to no more than 100 μL and may range from 10 to 50 μL in embodiments. The fluidic structures of the chip are subsequently loaded with said suspension, it being possible for this step to be automated. Inside the enrichment and reaction chamber, a temperature that is ideal for culture conditions, e.g., 37° C., is set by the heating element, so that the bacteria resistant to the active substance to be analyzed can multiply and form corresponding bacterial factors. MRSA strains, e.g., express the virulence factor of deoxyribonuclease, an enzyme which enzymatically catalyzes cleavage of DNA strands. This enzyme is released by the bacteria to the medium surrounding them. Simultaneously with the exponential propagation of the bacteria, the fluorescence-labeled substrate is enzymatically cleaved inside the enrichment and reaction chamber and, thus, the fluorophore is released. To obtain maximum enzymatic activity, the incubation temperature may be increased, depending on the temperature optimum of the enzyme, to 39° C., or to 45° C., 10 minutes prior to transporting the bulk phase on into the detection chamber 54. As a result, further acceleration of the test can be achieved.

After a defined time has elapsed, as a function of the bacterial count and species used, the contents of the chamber 52 are pumped on into the detection chamber 54, where the detached labels 70, i.e., the fluorescence, can be measured and quantified in a highly sensitive manner, for example by means of a photomultiplier 66. The retention device 60 located between the chambers 52 and 54 ensures that no particulate structures or bacteria 68, which may produce scattered-light signals, get into the detection chamber 54.

If no fluorescence is detectable, the active substance to be tested has an antibacterial action, it being possible for corresponding controls to be carried along. The incubation time taken by conventional methods for active-substance screening is largely undercut with the aid of this foil-based chip since the fluorescence signal can be detected in an extremely sensitive manner and, thus, a lot earlier than—as has been common so far—a turbidity of the medium that is caused by bacteria cells.

Instead of increasing the fluorescence intensity inside the detection chamber, it is alternatively also possible to measure the decrease in signals inside the enrichment and reaction chamber.

In addition to such a sensitivity test, embodiments may also comprise using such chips for verifying resistant bacteria, for example in foodstuffs. Here, incubation of the material to be tested (e.g., foodstuff samples, wash water of slaughterings or of herbs and vegetables) within a medium to which specific antibiotics are admixed (depending on which resistant bacteria are to be verified) may possibly be performed following enrichment of the bacteria, e.g., by means of particulate systems or filtration. Resistant bacteria will survive and can be verified on the chip.

FIG. 6 schematically represents a method of detecting the resistance of bacteria to an active substance to be analyzed in accordance with an embodiment.

A step 100 comprises introducing a culture medium containing an active substance to be analyzed and bacteria into the interaction chamber of a microfluidic chip comprising fluidic structures having a fluid inlet and an interaction chamber fluidically connected to the fluid inlet, and a labeled substrate that is immobilized inside the interaction chamber and is configured to enter into a verifiable interaction with bacterial factors of bacteria, a void volume of the fluidic structures not exceeding 100μ. A defined volume of culture medium may contain a defined bacterial count and a defined active substance in a defined concentration. Suspensions comprising corresponding culture medium volumes and corresponding counts of bacteria and different concentrations of active substance may be used successively or in parallel, for example to implement a concentration series for MIC/MBC determination. A step 102 comprises detecting information that is a measure of whether or not the verifiable interaction has taken place. A step 104 comprises outputting a display, on the basis of the detected information, as to whether there are resistant bacteria. This display may indicate, for example, whether the bacteria are resistant, intermediate, or sensitive to the active substance to be analyzed. The display may also indicate MIC and MBC.

Microfluidic chips, which may be foil-based, for screening antimicrobial active substances (e.g., against MRSA) may yield a result within a clearly shorter time period as compared to currently used methods (typically dilution tests). By using an extremely sensitive (and nevertheless cheap) SiPM able to detect individual photons, the fluorescence signal may be verifiable much earlier than the conventionally measured turbidity of the medium. The verification system is automatable (suitable for HTS) and is miniaturized, i.e., only very small amounts of active substance are used for the sensitivity test. The chip may be foil-based and therefore be manufactured at very low cost as a throw-away product in very high numbers. Labor-intensive cleaning steps may be dispensed with. In the above-described embodiment comprising a retention device inside the channel, particles and bacteria cells can be retained so that no scattering-light effects may occur in the detection, for example in the fluorescence measurement. Depending on the embodiment, integration of two or more copper mesh heaters (Cu mesh heaters) that can be thermostated differently enables incubation of the bacteria in the presence of the substance to be analyzed under optimum culture conditions (e.g., 37° C.), while the enzymatic reaction may proceed at a different optimum temperature (e.g., 45° C.). Alternatively, it is also possible to use two different temperatures one after the other inside the enrichment and reaction chamber. The increase in the reaction rate resulting therefrom further enables reduction of the measuring time. Since living bacteria are verified by using this method, the chip is also suitable for decontamination control.

Embodiments thus enable utilization of microfluidic chips in the search for novel, antimicrobial active substances with which infections can be treated, for example, which are caused by multi-resistant bacteria such as MRSA. In addition to MRSA, sensitivity tests on other bacteria are also included, it being possible for the chip to have an appropriate verification reaction implemented thereon. In addition to the application in the pharmaceutical industry, utilization in inspecting foodstuffs is also feasible. For example, wash water from slaughterhouses or of vegetables/salad/herbs can be examined, by using this system, as to whether there are resistant bacteria present. Once the bacteria have been enriched within a suitable filter and/or on a particle-based system, incubation is effected in the presence of a culture medium containing antibiotics and—just like in active-substance screening—verification of living bacteria within the microfluidic chip is effected.

Embodiments thus refer to a foil-based microfluidic chip for detecting living bacteria, e.g., for determining the sensitivity of bacterial germs. Embodiments thus relate to the use of a foil-based microfluidic chip for active-substance screening (e.g., in the pharmaceutical industry) and enable identifying, in a manner that is lower in cost and faster than conventional methods, which are typically based on turbidity measurements, new potential active substances for treating infections caused by resistant bacteria. In order to test the sensitivity of bacteria to a new potential active substance, a respective defined bacterial count of selected bacteria can be introduced into a culture medium volume, along with different concentrations of a new potential active substance, the antimicrobial properties of which are to be analyzed (e.g., as a concentration series), and can be examined as was described in order to determine MIC and MBC, for example.

Even though some aspects have been described within the context of a device, or the functionality of a device, it is understood that said aspects also represent features of a corresponding method, so that a block or a structural component of a device is also to be understood as a corresponding method step or as a feature of a method step. By analogy therewith, aspects that have been described within the context of or as a method step also represent a description of a corresponding functionality or block or detail or feature of a corresponding device.

While this invention has been described in terms of several embodiments, there are alterations, permutations, and equivalents which fall within the scope of this invention. It should also be noted that there are many alternative ways of implementing the methods and compositions of the present invention. It is therefore intended that the following appended claims be interpreted as including all such alterations, permutations and equivalents as fall within the true spirit and scope of the present invention.

1. A device for detecting the resistance of bacteria to an active substance to be analyzed, comprising:
a microfluidic chip comprising:
fluidic structures comprising a fluid inlet and an interaction chamber fluidically connected to the fluid inlet;
a labeled substrate that is immobilized inside the interaction chamber and is configured to enter into a verifiable interaction with bacterial factors of bacteria, so that by introducing a culture medium comprising an active substance to be analyzed and the bacteria into the interaction chamber and by detecting the result of the verifiable interaction, it can be ascertained whether the bacteria are resistant to the active substance to be analyzed,
a void volume of the fluidic structures not exceeding 100 μl;
a detector configured to detect information that is a measure of whether or not the verifiable interaction has taken place; and
an analyzer configured to output, on the basis of the detected information, a display with regard to the resistance of the bacteria to the active substance to be analyzed.

2. The device as claimed in claim 1, wherein the analyzer is configured to compare the detected information to at least one threshold value and to output a display, as a function of the comparison, as to whether or not the bacteria are resistant to the active substance to be analyzed, or wherein the analyzer is configured to compare the detected information to several threshold values and to output a display, as a function of the comparison, as to whether the bacteria are resistance, intermediate, or sensitive to the active substance to be analyzed.

3. The device as claimed in claim 1, wherein the microfluidic chip comprises a plurality of plastic foils, at least one of which is structured to define the fluidic structures.

4. The device as claimed in claim 1, wherein the substrate is labeled with a substance that is detached from the substrate by the interaction with the bacterial factors.

5. The device as claimed in claim 4, wherein the fluidic structures of the microfluidic module comprise a detection chamber fluidically coupled to the interaction chamber, a retention device being fluidically connected between the interaction chamber and the detection chamber, the retention device being configured to allow labels and/or labeled substrate fragments, which are detached from the labeled substrate on account of the interaction, to pass through and to retain the bacteria.

6. The device as claimed in claim 1, wherein the microfluidic chip further comprises an enrichment chamber fluidically connected between the fluid inlet and the interaction chamber.

7. The device as claimed in claim 6, comprising a heating element configured to heat contents of the enrichment chamber to a temperature suited for enriching the bacteria within the culture medium.

8. Device as claimed in claim 1, comprising a heating element configured to heat contents of the interaction chamber to a temperature suited for the verifiable interaction.

9. The device as claimed in claim 1, wherein the substrate is labeled with fluorescent dyes, absorption dyes or radioactive marked labels.

10. The device as claimed in claim 1, wherein the labeled substrate is mounted on a wall of the interaction chamber or on fibrous or particulate structures immobilized inside the interaction chamber.

11. The device as claimed in claim 1, wherein the microfluidic chip comprises several substrates labeled with different labels and immobilized inside one or more fluid chambers of the microfluidic chip.

12. The device as claimed in claim 11, wherein the labeled substrate is labeled with fluorophores or absorption dyes, and wherein the detector comprises a photomultiplier or a one-photon detector.

13. A method of detecting the resistance of bacteria to an active substance to be analyzed, comprising:
introducing a culture medium comprising an active substance to be analyzed and bacteria into the interaction chamber of a microfluidic chip which comprises fluidic structures comprising a fluid inlet and an interaction chamber fluidically connected to the fluid inlet, and comprises a labeled substrate immobilized inside the interaction chamber and being configured to enter into a verifiable interaction with bacterial factors of bacteria, a void volume of the fluidic structures not exceeding 100 μl;
detecting information that is a measure of whether or not the verifiable interaction has taken place; and
outputting a display, on the basis of the detected information, with regard to the resistance of the bacteria to the active substance to be analyzed.

14. The method as claimed in claim 13, comprising comparing the detected information to at least one threshold value and outputting the display, on the basis of the comparison, as to whether there are resistant bacteria.

15. The method as claimed in claim 14, comprising comparing the detected information to several threshold values and outputting the display, on the basis of the comparison, as to whether the bacteria are resistant, intermediate, or sensitive to the active substance to be analyzed.

16. The method as claimed in claim 14, the bacteria being known bacteria and the method being configured to perform an active-substance screening.
